(19) United States
(12) Patent Application Publication
(10) Pub. No.: US 2013/0217099 A1
(43) Pub. Date: Aug. 22, 2013

(54) Methods for Preventing, Removing, Reducing, or Disrupting Biofilm

(71) Applicants: Novozymes A/S, (US); Novozymes North America, Inc., (US)

(72) Inventors: Randy Deinhammer, Wake Forest, NC (US); Carsten Andersen, Vaerloese (DK)

(73) Assignees: Novozymes North America, Inc., Franklinton, NC (US); Novozymes A/S, Bagsvaerd (DK)

(21) Appl. No.: 13/863,030

(22) Filed: Apr. 15, 2013

Related U.S. Application Data
(63) Continuation of application No. 12/955,251, filed on Nov. 29, 2010.
Application No. 12/955,251 is a continuation of application No. 11/574,688, filed on Mar. 5, 2007, now abandoned.
(60) Provisional application No. 60/608,535, filed on Sep. 10, 2004.

(51) Int. Cl. C12N 9/24

(52) U.S. Cl. CPC C12N 9/2417
USPC 435/264

(57) ABSTRACT

The present invention relates to methods for preventing, removing, reducing, or disrupting biofilm present on a surface, comprising contacting the surface with an alpha-amylase derived from a bacterium.

### CROSS-REFERENCE TO RELATED APPLICATIONS

This application is a continuation of U.S. application Ser. No. 12/955,251 filed Nov. 29, 2010, which is a continuation of U.S. application Ser. No. 11/574,688 filed Mar. 5, 2007 (abandoned) which is a U.S.C. 371 national application of PCT/US2005/031813 filed Sep. 7, 2005, which claims priority or the benefit under 35 U.S.C. 119 of U.S. provisional application No. 60/608,535 filed Sep. 10, 2004, the contents of which are fully incorporated herein by reference.

### FIELD OF THE INVENTION

The present invention relates to improved methods of preventing, removing, reducing, or disrupting biofilm formation on a surface.

### DESCRIPTION OF THE RELATED ART

Biofilms are biological films that develop and persist at the surfaces of biotic or abiotic objects in aqueous environments from the adsorption of microbial cells onto the solid surfaces. This adsorption can provide a competitive advantage for the microorganisms since they can reproduce, are accessible to a wider variety of nutrients and oxygen conditions, are not washed away, and are less sensitive to antimicrobial agents. The formation of the biofilm is also accompanied by the production of exo-polymeric materials (polysaccharides, polyuronic acids, alginates, glycoproteins, and proteins) which together with the cells form thick layers of differentiated structures separated by water-filled spaces. The resident microorganisms may be individual species of microbial cells or mixed communities of microbial cells, which may include aerobic and anaerobic bacteria, algae, protozoa, and fungi. Thus, the biofilm is a complex assembly of living microorganisms embedded in an organic structure composed of one or more matrix polymers which are secreted by the resident microorganisms.

Biofilms can develop into macroscopic structures several millimeters or centimeters in thickness and cover large surface areas. These formations can play a role in restricting or entirely blocking flow in plumbing systems, decreasing heat transfer in heat exchangers, or causing pathogenic problems in municipal water supplies, food processing, medical devices (e.g., catheters, orthopedic devices, implants, endoscopes). Moreover, biofilms often decrease the life of materials through corrosive action mediated by the embedded microorganisms. This biological fouling is a serious economic problem in industrial water process systems, pulp and paper production processes, cooling water systems, injection wells for oil recovery, cooling towers, porous media (sand and soil), marine environments, and air conditioning systems, and any closed water recirculation system. Biofilms are also a severe problem in medical science and industry causing dental plaque, infections (Costerton et al., 1999, Science 284: 1318-1322), contaminated endoscopes and contact lenses, prosthetic device colonisation and biofilm formation on medical implants.

The removal or prevention of biofilm traditionally requires the use of dispersants, surfactants, detergents, enzyme formulations, anti-microbials, biocides, boil-out procedures, and/or corrosive chemicals, e.g., base. Procedures for using these measures are well known in the art. For example, removal of biofilm built-up in a paper machine in the pulp and paper industry traditionally requires a deposit control program including proper housekeeping to keep surfaces free of splashed stock, anti-microbial treatment of fresh water and additives, the use of biocides to reduce microbiological growth on the machine, and scheduled boil-outs to remove the deposits that do form.

Bacteria growing in biofilms are more resistant to antibiotics and disinfectants than planktonic cells and the resistance increases with the age of the biofilm. Bacterial biofilm also exhibits increased physical resistance towards desiccation, extreme temperatures or light. As mentioned, biofilm formation causes industrial, environmental and medical problems and the difficulties in cleaning and disinfection of bacterial biofilm with chemicals is a major concern in many industries. Furthermore, the trend towards milder disinfection and cleaning compositions to reduce their environmental impact may increase the insufficient cleaning of surfaces covered with biofilm.

It is an object of the present invention to provide improved methods for preventing or removing biofilm present on a surface.

### BRIEF DESCRIPTION OF THE DRAWING

FIG. 1 shows a comparison of the percentage (%) of total hydrolysis of raw wheat starch for three different alpha-amylases.

FIG. 2 is a chromatogram comparing 1) Alpha-amylase A, 2) detergent alone 3) Alpha-amylase C biofilm removal.

### SUMMARY OF THE INVENTION

The present invention relates to methods for preventing, removing, reducing, or disrupt biofilm formation on a surface, comprising contacting the surface with alpha-amylase derived from a bacterium.

The term “surface” is defined herein as any surface which may be covered by biofilm or is prone to biofilm formation. Examples of surfaces may be any hard surface such as metal, plastics, rubber, board, glass, wood, paper, concrete, rock, marble, gypsum and ceramic materials, which optionally are coated, for example, with paint or enamel; any soft surface such as fibers of any kind (e.g., yarns, textiles, vegetable fibers, rock wool, and hair); or any porous surfaces; skin (human or animal); keratinous materials (e.g., nails); and internal organs (e.g., lungs). The hard surface can be present as a part of a cooling tower, water treatment plant, water tanks, dairy, food processing plant, chemical or pharmaceutical process plant, or medical device (e.g., catheters, orthopedic devices, implants). The porous surface can be present in a filter, e.g., a membrane filter.

The term “effective amount” is defined herein as the amount of one or more alpha-amylases that is sufficient to degrade a microbially-produced biofilm comprising alpha-1,4 glucosidic linkages. The effective amount of the one or more alpha-amylase will depend on factors including: the alpha-amylase(s) in question, whether the aim is preventing, removing, or reducing biofilms present on a surface, the period of time desirable for, e.g., degrading a microbially-produced biofilm. High amounts/concentrations of enzyme(s) will in general require shorter times of treatment, while low amounts/concentrations longer times. Further, for instance, preventing biofilm on a surface prone to biofilm formation will in general require lower amounts/concentrations of enzyme(s) than the actual removal of biofilm from a corresponding contaminated surface. However, typical effective usage levels are between 0.005 to 500 mg of alpha-amylase protein per L biofilm control solution, preferably between 0.01-100 mg of enzyme protein per L biofilm control solution. The term “biofilm control solution” refers to a solution used according to the invention for preventing, removing, reducing or disrupting biofilm present on a surface. The method of the invention may result in 10-10⁸-fold, preferably 10³-10⁶-fold biofilm reduction in terms of average plate count under the conditions indicated in Example 4 below.

### DETAILED DESCRIPTION OF THE INVENTION

The present invention relates to improved methods of preventing, removing, or reducing biofilms present on a surface, comprising contacting the surface with an effective amount of an alpha-amylase as defined below. The methods of the present invention may be used to prevent, remove, reduce, or disrupt biofilm formation on a surface. One of ordinary skill in the art will recognize that such methods may be employed at different stages of biofilm formation.

By using an alpha-amylase in an effective amount of surfaces improved biofilm prevention and/or removal is obtained, especially in those instances where some of the microbes present in the biofilm produce alpha-1,4 linked glucose polysaccharides such as amylose, amylopectin, mixtures of these two polysaccharides (i.e., starch), and glycogen.

In the first aspect the invention relates to a method for preventing or removing biofilm on a surface, comprising contacting the surface with an alpha-amylase derived from a bacterium. In a preferred embodiment the bacterial alpha-amylase is derived from a strain of Bacillus.

### Alpha-Amylase

The alpha-amylase used according to the invention is derived from a bacterium, preferably derived from a strain of Bacillus sp., especially selected from the group consisting of: the AA560 alpha-amylase disclosed as SEQ ID NO: 2 in WO 00/60060 (SEQ ID NO: 2 herein), the Bacillus flavothermus disclosed in U.S. patent application Ser. No. 10/877,847, Bacillus sp. alpha-amylases disclosed in WO 95/26397, alpha-amylases derived from Bacillus sp. NCIB 12289, NCIB 12512, NCIB 12513, DSM 9375, DSMZ no. 12649, KSM AP1378 (WO 97/00324), KSM K36 or KSM K38 (EP 1,022,334), and the #707 alpha-amylase disclosed by Tsukamoto et al., Biochemical and Biophysical Research Communications, 151 (1988), pp. 25-31.

In a preferred specific embodiment of the invention the alpha-amylase is the AA560 alpha-amylase shown in SEQ ID NO: 2 herein and/or the AMY1048 alpha-amylase shown in SEQ ID NO: 4 herein, or an alpha-amylase having a degree of identity of at least 60%, preferably at least 70%, more preferred at least 80%, even more preferred at least 90%, such as at least 95%, at least 96%, at least 97%, at least 98% or at least 99% to any of the sequences shown in SEQ ID NOS: 2 or 4 herein.

In a preferred embodiment the alpha-amylase used is a variant of the parent alpha-amylase disclosed in SEQ ID NO: 2 herein having a deletion in positions D183 and/or G184, preferably wherein said alpha-amylase variant further has a substitution in or corresponding to position N195F, especially wherein the parent alpha-amylase has one or more of the following deletions/substitutions in SEQ ID NO: 2 herein: Delta (R81-G182); Delta (D183-G184); Delta (D183-G184)+N195F; R181Q+N445Q+K446N; Delta (D183-G184)+R181Q, Delta (D183-G184) and one or more of the following substitutions: R118K, N195F, R320K, R458K, especially wherein said parent alpha-amylase has the following mutations:

Delta (D183+G184)+R118K+N195F+R320K+R458K (i.e., in SEQ ID NO: 2 herein).

In another preferred embodiment the alpha-amylase is the AA560 alpha-amylase shown in SEQ ID NO: 2 or a variant thereof further comprising one or more of the following substitutions: M9L, M202L, V214T, M323T, M382Y or M9L, M202L, V214T, M323T and E345R.

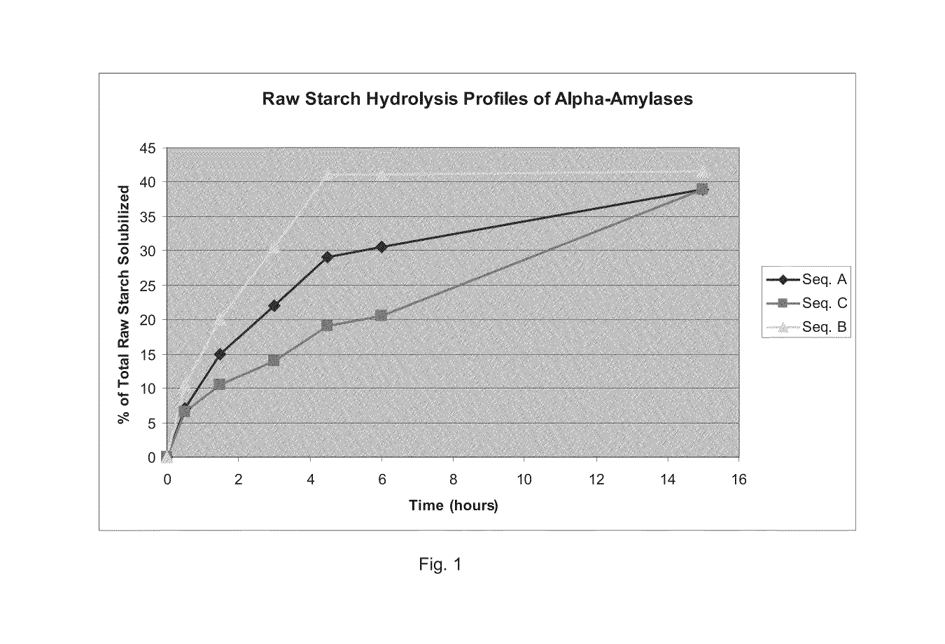

In a preferred embodiment the alpha-amylase has a percentage (%) of hydrolyzed raw starch that is higher than 15, preferably 25, especially 35, after 5 hours at 40° C., 3 mg enzyme protein per g starch, pH 8.0 (See Example 2 and FIG. 1).

In another preferred embodiment the alpha-amylase comprises Asn-Gly-Thr-Met-Met-Gln-Tyr-Phe-Glu-Trp in its N-terminal amino acid region. Examples of such alpha-amylase include Alpha-Amylase A and Alpha-Amylase B used in Example 2.

In an embodiment the alpha-amylase is derived from a strain of Bacillus licheniformis with the sequence shown as SEQ ID NO: 6 herein, or an alpha-amylase having a degree of identity of at least 60%, preferably at least 70%, more preferred at least 80%, even more preferred at least 90%, such as at least 95%, at least 96%, at least 97%, at least 98% or at least 99% to any of the sequences shown in SEQ ID NO: 6 herein, preferably with a substitution in a position corresponding to position M197, preferably M197L, T, I, N, D, Q, E,P,W, especially M197L or T.

Commercially available alpha-amylase products or products comprising alpha amylases include product sold under the following trade names: STAINZYME™, DURAMYL™ (Novozymes A/S, Denmark), BIOAMYLASE D(G), BIOAMYLASE™ L (Biocon India Ltd.), KEMZYM™ AT 9000(Biozym Ges. m.b.H, Austria), PURASTAR™ ST, PURASTAR™ HPAmL, PURAFECT™ OxAm, RAPIDASE™ TEX (Genencor Int. Inc, USA), KAM (KAO, Japan).

In a preferred embodiment, surfaces prone to biofilm formation may be subjected to the methods of the present invention as a preventative measure prior to any biofilm formation so no biofilm forms. Alternatively, at the first indication of biofilm formation, the methods may be used to prevent further formation and to remove the biofilm that has deposited on a surface. Furthermore, in situations where there is a heavy build-up of biofilm on a surface, the methods may be used to reduce the level of biofilm or to remove it partially or completely.

A biofilm may comprise an integrated community of one or two or more microorganisms or predominantly a specific microorganism (Palmer and White, 1997, Trends in Microbiology 5: 435-440; Costerton et al., 1987, Annual Reviews of Microbiology 41: 435-464; Mueller, 1994, TAPPI Proceedings, 1994 Biological Sciences Symposium 195-201). In the methods of the present invention, the one or more microorganisms may be any microorganism involved in biofilm formation including, but not limited to, aerobic bacteria or anaerobic bacteria (Gram positive and Gram negative), fungi (yeast or filamentous fungus), algae, and/or protozoa. Contemplated bacteria include bacteria selected from the group consisting of: Pseudomonas spp. including Pseudomonas aeruginosa, Azotobacter vinelandii, Escherichia coli, Corynebacterium diphteriae, Clostridium botulinum, Streptococcus spp, Acetobacter, Leuconostoc, Betabacterium, Pneumococci, Mycobacterium tuberculosis, Aeromonas, Burkholderie, Flavobacterium, Salmonella, Staphylococcus.

In a preferred embodiment, the microorganism is an aerobic bacterium. In a more preferred embodiment, the aerobic bacterium is an Aeromonas strain. In another more preferred embodiment, the aerobic bacterium is a Burkholderie strain. In another more preferred embodiment, the aerobic bacterium is a Flavobacterium strain. In another more preferred embodiment, the aerobic bacterium is a Microbacterium strain. In another more preferred embodiment, the aerobic bacterium is a Pseudomonas strain. In another more preferred embodiment, the aerobic bacterium is a Salmonella strain. In another more preferred embodiment, the aerobic bacterium is a Staphylococcus strain. In another more preferred embodiment, the aerobic bacterium is from the family Enterobacteriaceae (including e.g., Escherichia coli).

In a most preferred embodiment, the aerobic bacterium is Burkholderie cepacia. In another most preferred embodiment, the aerobic bacterium is a Microbacterium imperiale or Mycobacterium tuberculosis. In another most preferred embodiment, the aerobic bacterium is Pseudomonas aeruginosa. In another most preferred embodiment, the aerobic bacterium is Pseudomonas fluorescens. In another most preferred embodiment, the aerobic bacterium is Pseudomonas oleovorans. In another most preferred embodiment, the aerobic bacterium is Pseudomonas pseudoalcaligenes. In another most preferred embodiment, the aerobic bacterium is Salmonella enteritidis. In another most preferred embodiment, the aerobic bacterium is Staphylococcus aureus. In another most preferred embodiment, the aerobic bacterium is Staphylococcus epidermidis.

In another preferred embodiment, the microorganism is an anaerobic bacteria. In another more preferred embodiment, the anaerobic bacterium is a Desulfovibrio strain. In another most preferred embodiment, the anaerobic bacterium is Desulfovibrio desulfuricans.

In another preferred embodiment, the microorganism is a fungus such as a yeast or filamentous fungus. In another more preferred embodiment, the yeast is a Candida strain. In another most preferred embodiment, the yeast is Candida albicans.

As mentioned above the treatment time for preventing or removing biofilm will depend on the dosage of the alpha-amylase, and the level of biofilm on the surface or prone to the area in question, but should preferably be adapted to the time normally used for conventional treatment of biofilm with antibiotics, biocides, bactericides, fungicides, bleaching agents, surfactants, caustic, and/or biopolymer degrading agents. Consequently, the dosage of the alpha-amylase may be adjusted according to the time period used during conventional treatments. However, where the alpha-amylase treatment is a separate step in the processing, the dosage of the alpha-amylase used will depend on the time period desired to accomplish the treatment.

In terms of alpha-amylase activity, the appropriate dosage of alpha-amylase for preventing or removing biofilms will depend on the amount of biofilm on the surface or prone to the area in question. The skilled person may determine a suitable alpha-amylase unit dosage. The dosage may be expressed in alpha-amylase units. Alpha-amylase units may be determined as “KNU”, using the assay described below in the “Materials & Methods”-section. Biofilm contaminated or prone areas are preferably treated for between 1 minute and 2 days, preferably between 10 minutes and 1 day, preferably between 1 hour and 15 hours, more preferably less that 10 hours, with an alpha-amylase dosage of between 0.005 to 500 mg of alpha-amylase protein per L biofilm control solution, preferably between 0.01 to 100 mg of alpha-amylase protein per L biofilm control solution.

The alpha-amylase may be part of a composition to be used in the methods of the present invention. The composition may be in any form suitable for the use in question, e.g., in the form of a dry powder, agglomerated powder, or granulate, in particular a non-dusting granulate, liquid, in particular a stabilized liquid, or protected alpha-amylase. Granulates and agglomerated powders may be prepared by conventional methods, e.g., by spraying the alpha-amylase onto a carrier in a fluid-bed granulator. The carrier may consist of particulate cores having a suitable particle size. The carrier may be soluble or insoluble, e.g., a salt (such as sodium chloride or sodium sulfate), sugar (such as sucrose or lactose), sugar alcohol (such as sorbitol), or starch. The alpha-amylase may be contained in slow-release formulations. Methods for preparing slow-release formulations are well known in the art. Liquid alpha-amylase preparations may, for instance, be stabilized by adding nutritionally acceptable stabilizers such as a sugar, sugar alcohol, or another polyol, and/or lactic acid or another organic acid according to established methods.

The composition may be augmented with one or more agents for preventing or removing the formation of the biofilm. These agents may include, but are not limited to, dispersants, surfactants, detergents, other enzymes, anti-microbials, and biocides.

In a preferred embodiment, the agent is a surfactant. The surfactant may be a non-ionic including semi-polar and/or anionic and/or cationic and/or zwitterionic surfactant.

Anionic surfactants contemplated include linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl- or alkenylsuccinic acid or soap.

Non-ionic surfactants contemplated include alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl N-alkyl derivatives of glucosamine (“glucamides”).

The surfactants may be present at a level of from 0.1% to 60% by weight of the enzyme biofilm removal composition.

In a more preferred embodiment, the surfactant is sodium dodecyl sulfate, quaternary ammonium compounds, alkyl pyridinium iodides, Tween 80, Tween, 85, Triton X-100, Brij 56, biological surfactants, rhamnolipid, surfactin, visconsin, or sulfonates.

The formation of biofilm is generally accompanied by the production of exo-polymeric materials (polysaccharides, polyuronic acids, alginates, glycoproteins, and proteins) which together with the cells form thick layers of differentiated structures separated by water-filled spaces (McEldowney and Fletcher, 1986, Journal of General Microbiology 132: 513-523; Sutherland, Surface Carbohydrates of the Prokaryotic Cell, Academic Press, New York, 1977, pp. 27-96). In the methods of the present invention, the alpha-amylase composition may further comprise one or more other enzymes capable of degrading the exo-polymeric materials such as polysaccharides, polyuronic acids, alginates, glycoproteins, and proteins.

### Other Enzyme Activities

In a preferred embodiment, the one or more other enzymes may be selected from the group consisting of an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, oxididases, including carbohydrate oxidases, peroxidases, laccase, lipase, mannosidase, pectinolytic enzyme, peptidoglutaminase, phytase, polyphenoloxidase, proteolytic enzyme, ribonuclease, transglutaminase, or xylanase.

The other enzyme(s) may be selected according to the properties of the specific biofilm which is to be removed, or a combination of several enzymes having different enzyme activities may be used.

In a preferred embodiment, the other enzyme is selected from the group consisting of 1,2-1,3-alpha-D-mannan mannohydrolase, 1,3-beta-D-xylan xylanohydrolase, 1,3-beta-D-glucan glucanohydrolase, 1,3(1,3;1,4)-alpha-D-glucan 3-glucanohydrolase, 1,3(1,3;1,4)-beta-D-glucan 3(4)-glucanohydrolase, 1,3-1,4-alpha-D-glucan 4-glucanohydrolase, 1,4-alpha-D-glucan glucanehydrolase, 1,4-alpha-D-glucan glucohydrolase, 1,4-(1,3:1,4)-beta-D-glucan 4-glucanohydrolase, 1,4-beta-D-glucan glucohydrolase, 1,4-beta-D-xylan xylanohydrolase, 1,4-beta-D-mannan mannanohydrolase, 1,5-alpha-L-arabinan 1,5-alpha-L-arabinanohydrolase, 1,4-alpha-D-glucan maltohydrolase, 1,6-alpha-D-glucan 6-glucanohydrolase, 2,6-beta-D-fructan fructanohydrolase, alpha-Dextrin 6-glucanohydrolase, alpha-D-galactoside galactohydrolase, alpha-D-glucoside glucohydrolase, alpha-D-mannoside mannohydrolase, acylneuraminyl hydrolase, Aerobacter-capsular-polysaccharide galactohydrolase, beta-D-fructofuranoside fructohydrolase, beta-D-fucoside fucohydrolase, beta-D-fructan fructohydrolase, beta-D-galactoside galactohydrolase, beta-D-glucoside glucohydrolase, beta-D-glucuronoside glucuronosohydrolase, beta-D-mannoside mannohydrolase, beta-N-acetyl-D-hexosaminide N-acetylhexosamino hydrolase, cellulose-sulfate sulfohydrolase, collagenase, dextrin 6-alpha-D-glucanohydrolase, glycoprotein-phosphatidylinositol phosphatidohydrolase, hyaluronate 4-glycanohydrolase, hyaluronoglucuronidase, pectin pectylhydrolase, peptidoglycan N-acetylmuramoylhydrolase, phosphatidylcholine 2-acylhydrolase, phosphatidylcholine 1-acylhydrolase, poly(1,4-alpha-D-galacturonide)galacturonohydrolase, poly(1,4-(N-acetyl-beta-D-glucosaminide))-glycanohydrolase, sucrose alpha-glucosidase, triacylglycerol acylhydrolase, and triacylglycerol protein-acylhydrolase.

### Proteolytic Enzyme

The other enzyme may be any enzyme having proteolytic activity under the actual process conditions. Thus, the enzyme may be a proteolytic enzyme of plant origin, e.g., papain, bromelain, ficin, or of animal origin, e.g., trypsin and chymotrypsin, or of microbial origin, i.e., bacterial, yeast, or filamentous fungal. It is understood that any mixture of various proteolytic enzyme may be applicable in the process of the invention.

In another preferred embodiment, the other enzyme is a proteolytic enzyme such as a serine protease, a metalloprotease, or an aspartate protease.

A sub-group of the serine proteases are commonly designated as subtilisins. A subtilisin is a serine protease produced by Gram-positive bacteria or fungi. The amino acid sequence of a number of subtilisins have been determined, including at least six subtilisins from Bacillus strains, namely, subtilisin 168, subtilisin BPN, subtilisin Carlsberg, subtilisin DY, subtilisin amylosacchariticus, and mesentericopeptidase, one subtilisin from an actinomycetales, thermitase from Thermoactinomyces vulgaris, and one fungal subtilisin, proteinase K from Tritirachium album. A further subgroup of the subtilisins, subtilases, has been recognised more recently. Subtilases are described as highly alkaline subtilisins and comprise enzymes such as subtilisin PB92 (MAXACAL®, Gist-Brocades NV), subtilisin 309 (SAVINASE®, Novozymes A/S), and subtilisin 147 (ESPERASE®, Novozymes A/S).

A “subtilisin variant or mutated subtilisin protease” is defined herein as a subtilisin that has been produced by an organism which is expressing a mutant gene derived from a parent microorganism which possessed an original or parent gene and which produced a corresponding parent enzyme, the parent gene having been mutated in order to produce the mutant gene from which said mutated subtilisin protease is produced when expressed in a suitable host. These mentioned subtilisins and variants thereof constitute a preferred class of proteases which are useful in the method of the invention. An example of a useful subtilisin variant is a variant of subtilisin 309 (SAVINASE®) wherein, in position 195, glycine is substituted by phenylalanine (G195F or ¹⁹⁵Gly to ¹⁹⁵Phe).

Commercially available proteases may be used in the methods of the present invention. Examples of such commercial proteases are ALCALASE® (produced by submerged fermentation of a strain of Bacillus licheniformis), ESPERASE® (produced by submerged fermentation of an alkalophilic species of Bacillus), RENNILASE® (produced by submerged fermentation of a non-pathogenic strain of Mucor miehei), SAVINASE® (produced by submerged fermentation of a genetically modified strain of Bacillus), e.g., the variants disclosed in the International Patent Application published as WO 92/19729, and DURAZYM® (a protein-engineered variant of SAVINASE®), POLARZYME™, EVERLASE™. All the above-mentioned commercial proteases are available from Novozymes A/S, DK-2880 Bagsværd, Denmark.

Other preferred serine proteases are proteases from Aspergillus, Bacillus such as Bacillus alcalophilus, Bacillus cereus, Bacillus vulgatus, Bacillus mycoide, Rhizopus, and subtilins from Bacillus, especially proteases from the speciesNocardiopsis such as Nocardiopsis natto Nocardiopsis dassonvillei (see, WO 88/03947), especially proteases from the species Nocardiopsis sp. NRRL 18262, and Nocardiopsis dassonvillei NRRL 18133. Yet other preferred proteases are the serine proteases from mutants of Bacillus subtilisins disclosed in the International Patent Application No. PCT/DK89/00002 and WO 91/00345, and the proteases disclosed in EP 415 296.

Another preferred class of proteases is the metalloproteases of microbial origin. Conventional fermented commercial metalloproteases may be used in the methods of the present invention such as is NEUTRASE® (Zn) (produced by submerged fermentation of a strain of Bacillus subtilis), available from Novozymes A/S, DK-2880 Bagsværd, Denmark; BACTOSOL® WO and BACTOSOL® SI, available from Sandoz AG, Basle, Switzerland; TOYOZYME®, available from Toyo Boseki Co. Ltd., Japan; and PROTEINASE K® (produced by submerged fermentation of a strain of Bacillus sp. KSM-K16), available from Kao Corporation Ltd., Japan.

The protease may be used in a dosage of between 0.005 to 500 mg enzyme protein per L biofilm control solution, preferably between 0.01 to 100 mg enzyme protein per L biofilm control solution.

### Lipases

In another preferred embodiment, the other enzyme is a lipase, especially a microbial lipase. As such, the lipase may be selected from yeast, e.g., Candida; bacteria, e.g., Pseudomonas or Bacillus; or filamentous fungi, e.g., Humicola or Rhizomucor. More specifically, suitable lipases may be the Rhizomucor miehei lipase (e.g., prepared as described in EP 238 023), Thermomyces lanuginosa lipase e.g., prepared as described in EP 305 216, Humicola insolens lipase, Pseudomonas stutzeri lipase, Pseudomonas cepacia lipase, Candida antarctica lipase A or B, or lipases from rGPL, Absidia blakesleena, Absidia corymbifera, Fusarium solani, Fusarium oxysporum, Penicillum cyclopium, Penicillum crustosum, Penicillum expansum, Rhodotorula glutinis, Thiarosporella phaseolina, Rhizopus microsporus, Sporobolomyces shibatanus, Aureobasidium pullulans, Hansenula anomala, Geotricum penicillatum, Lactobacillus curvatus, Brochothrix thermosohata, Coprinus cinerius, Trichoderma harzanium, Trichoderma reesei, Rhizopus japonicus, or Pseudomonas plantari. Other examples of suitable lipases may be variants of any one of the lipases mentioned above, e.g., as described in WO 92/05249 or WO 93/11254.

Examples of commercially available lipases include: LIPOLASE™, LIPOLASE ULTRA™, LIPOPRIME™, LIPEX™ from Novozymes, Denmark).

The lipase may be used in a dosage of between 0.005 to 500 mg enzyme protein per L biofilm control solution, preferably between 0.01 to 100 mg enzyme protein per L biofilm control solution.

### Cellulases

In another preferred embodiment, the other enzyme is a cellulase or cellulolytic enzyme, which refers to an enzyme which catalyses the degradation of cellulose to glucose, cellobiose, triose and other cellooligosaccharides. Preferably, the cellulase is an endoglucanase, more preferably a microbial endoglucanase, especially a bacterial or fungal endoglucanase. Examples of bacterial endoglucanases are endoglucanases obtained from or producible by bacteria from the group of genera consisting of Pseudomonas or Bacillus lautus.

The cellulase or endoglucanase may be an acid, neutral, or alkaline cellulase or endoglucanase, i.e., exhibiting maximum cellulolytic activity in the acid, neutral or alkaline pH range, respectively. Accordingly, a useful cellulase or endoglucanase is an acid cellulase or endoglucanase, preferably a fungal acid cellulase or endoglucanase, more preferably a fungal acid cellulase or endoglucanse enzyme with substantial cellulolytic activity under acidic conditions, which is obtained from or producible by fungi from the group consisting of Trichoderma, Actinomyces, Myrothecium, Aspergillus, and Botrytis.

A preferred acid cellulase or endoglucanase is obtained from the group consisting of Aspergillus niger, Aspergillus oryzae, Botrytis cinerea, Myrothecium verrucaria, Trichoderma longibrachiatum, Trichoderma reesei, and Trichoderma viride.

Another useful cellulase or endoglucanase is a neutral or alkaline cellulase or endoglucanse, preferably a fungal neutral or alkaline cellulase or endoglucanse, more preferably a fungal alkaline cellulase or endoglucanase with substantial cellulolytic activity under alkaline conditions, which is obtained from fungi selected from the group consisting of Acremonium, Aspergillus, Chaetomium, Cephalosporium, Fusarium, Gliocladium, Humicola, Irpex, Myceliophthora, Mycogone, Myrothecium, Papulospora, Penicillium, Scopulariopsis, Stachybotrys, and Verticillium.

A preferred alkaline cellulase or endoglucanase is obtained from the group consisting of Cephalosporium sp., Fusarium oxysporum, Humicola insolens, or Myceliopthora thermophila, or preferably from the group consisting of Cephalosporium sp., RYM-202, Fusarium oxysporum, DSM 2672, Humicola insolens, DSM 1800, or Myceliopthora thermophila, CBS 117.65.

In another preferred embodiment, the other enzyme is a xylanase such as an endo-1,3-beta-xylosidase (EC 3.2.1.32), xylan 1,4-beta-xylosidase (EC 3.2.1.37), and alpha-L-arabinofuranosidase (EC 3.2.1.55). Preferably the xylanase is obtained from Aspergillus aculeatus (an enzyme exhibiting xylanase activity, which enzyme is immunologically reactive with an antibody raised against a purified xylanase derived from Aspergillus aculeatus CBS 101.43, see, for example, WO 94/21785); Aspergillus oryzae (see, for example, SU 4610007); Aureobasidium pullulans (see, for example, EP 0 373 107 A2); Bacillus circulans (WO 91/18978); Bacillus pumilus (see, for example, WO 92/03540); Bacillus stearothermophilus (see, for example, WO 91/18976, WO 91/10724); Bacillus sp. AC13 (especially the strain NCIMB 40482, see, for example, WO 94/01532); Humicola insolens (see, for example, WO 92/17573); Rhodothermus (see, for example, WO 93/08275); Streptomyces lividans (see, for example, WO 93/03155); Streptomyces viridosporus (see, for example, EP 496 671 A); Bacillus licheniformis (see, for example, JP 9213868); Thermoascus aurantiacus (see, for example, U.S. Pat. No. 4,966,850); Trichoderma longibrachiatum and Chainia sp. (see, for example, EP 0 353 342 A1); Trichoderma harzianum and Trichoderma reseei (see, for example, U.S. Pat. No. 4,725,544); Thermomyces lanuginosus (see, for example, EP 0 456 033 A2); Thermomonospora fusca (see, for example, EP 0 473 545 A2); Trichoderma longibrachiatum (see W. J. J. van den Tweel et al., Eds., Stability of Enzymes, Proceedings of an International Symposium held in Maastrich, The Netherlands, 22-25 Nov. 1992, Fisk, R. S. and Simpson, pp. 323-328); Dictyoglomus (see, for example, WO 92/18612); Streptomyces (see, for example, U.S. Pat. No. 5,116,746); and/or Thermotoga (see, for example, WO 93/19171). Other examples of suitable xylanases may be variants (derivatives or homologues) of any one of the above-noted enzymes having xylanolytic activity.

### Examples of commercially available cellulase containing products include NOVOZYM™ 342, CELLUZYME™, CAREZYME™, RENOZYME™ (all Novozymes, Denmark).

The cellulase may be used in a dosage of between 0.005 to 500 mg enzyme protein per L biofilm control solution, preferably between 0.01 to 100 mg enzyme protein per L biofilm control solution.

### Pectinases

In another preferred embodiment, the other enzyme is a pectinase such as a polygalacturonase (EC 3.2.1.15), pectinesterase (EC 3.2.1.11), or pectin lyase (EC4.2.2.10). A suitable source organism for pectinases may be Aspergillus niger.

In another preferred embodiment, the other enzyme in the alpha-amylase composition comprises a hydrolytic enzyme composition produced by a strain of the fungus Aspergillus aculeatus, preferably Aspergillus aculeatus, CBS 101.43. It is known that this strain produces an enzyme composition comprising pectinolytic and a range of hemicellulolytic enzyme activities.

Examples of commercially available pectinase containing products include: BioPrep™, SCOURZYME™ and PECTAWASH™ (Novozymes, Denmark).

The pectinase may be used in a dosage of between 0.005 to 500 mg enzyme protein per L biofilm control solution, preferably between 0.01 to 100 mg enzyme protein per L biofilm control solution.

### Oxidoreductase

In another embodiment of the invention the alpha-amylase is combined with an oxidoreductase, such as an oxidase, peroxidase, or laccase.

a) Laccases act on molecular oxygen and yield water (H₂O) without any need for peroxide (e.g. H₂O₂),
b) Oxidases act on molecular oxygen (O₂) and yield peroxide (H₂O₂), and
c) Peroxidases act on peroxide (e.g. H₂O₂) and yield water (H₂O).

Examples of laccases (E.C. 1.10.3.2) include laccases derived from a strain of Polyporus sp., in particular a strain of Polyporus pinsitus or Polyporus versicolor, or a strain of Myceliophthora sp., in particular M. thermophila, a strain of Scytalidium sp., in particular S. thermophilium, a strain of Rhizoctonia sp., in particular Rhizoctonia praticola or Rhizoctonia solani, or a strain of a Rhus sp., in particular Rhus vernicifera. The laccase may also be derived from a fungus such as Collybia, Fomes, Lentinus, Pleurotus, Aspergillus, Neurospora, Podospora, Phlebia, e.g. P. radiata (WO 92/01046), Coriolus sp., e.g., C. hirsitus (JP 2-238885), or Botrytis.

In specifically contemplated embodiments, the laccase may be selected from the group consisting of: the Polyporus pinisitus laccase (also called Trametes villosa laccase) described in WO 96/00290, the Myceliophthora thermophila laccase described in WO 95/33836, the Scytalidium thermophilium laccase described in WO 95/33837, the Pyricularia oryzae laccase which can be purchased from SIGMA under the trade name SIGMA no. L5510, the Coprinus cinereus laccase described in WO 96/06930, and the Rhizoctonia solani laccase described WO 95/07988.

Examples of peroxidases (1.11.1.7) include peroxidases derived from plants (e.g., horseradish peroxidase) or micro-organisms including fungi and bacteria, such as a strain of Coprinus sp., such as Coprinus cinereus or Coprinus macrorhizus, or bacteria such as Bacillus, such as Bacillus pumilus.

In specifically contemplated embodiments, the peroxidase may be selected from the group consisting of: the Coprinus cinereus IF08371 peroxidase or variants thereof described in WO 95/10602, and the haloperoxidase originating from a strain of Curvularia verruculosa CBS 147.63 described in WO 97/04102.

Contemplated oxidases include especially carbohydrate oxidases, which are enzymes classified under EC 1.1.3. Carbohydrate oxidases include glucose oxidase (E.C. 1.1.3.4), hexose oxidase (E.C. 1.1.3.5) xylitol oxidase, galactose oxidase (E.C. 1.1.3.9), pyranose oxidase (E.C. 1.1.3.10), alcohol oxidase (E.C. 1.1.3.13).

Carbohydrate oxidases may be derived from any origin, including, bacterial, fungal, yeast or mammalian origin.

Examples of glucose oxidases include glucose oxidases derived from Aspergillus sp., such as a strain of Aspergillus niger, or from a strain of Cladosporium sp. in particular Cladosporium oxysporum, especially Cl. oxysporum CBS 163 described in WO 95/29996.

Examples of hexose oxidases include hexose oxidases produced by the red sea-weed Chondrus crispus (commonly known as Irish moss) (Sullivan and Ikawa, (1973), Biochim. Biophys. Acts, 309, p. 11-22; Ikawa, (1982), Meth. in Enzymol. 89, carbohydrate metabolism part D, 145-149) that oxidizes a broad spectrum of carbohydrates and the red sea-weed Iridophycus flaccidum that produces easily extractable hexose oxidases, which oxidize several different mono- and disaccharides (Bean and Hassid, (1956), J. Biol. Chem, 218, p. 425; Rand et al. (1972, J. of Food Science 37, p. 698-710).

The oxidoreductase may be used in a dosage of between 0.005 to 500 mg enzyme protein per L biofilm control solution, preferably between 0.01 to 100 mg enzyme protein per L biofilm control solution.

In a final aspect the invention relates to the use of a Bacillus alpha-amylase for preventing, removing, reducing, or disrupting biofilm formation on a surface. In a preferred embodiment the alpha-amylase is a Bacillus alpha-amylase, preferably one mentioned above in the “Alpha-Amylase” section.

The present invention is further described by the following examples which should not be construed as limiting the scope of the invention.

### Materials & Methods

Chemicals used as buffers and reagents were commercial products of at least reagent grade.

### Enzymes:

Alpha-amylase A is a variant alpha-amylase of the parent Bacillus sp. alpha-amylase disclosed as SEQ ID NO: 2 in WO 00/60060. The amino acid sequence of said alpha-amylase has the following six amino acid deletions/substitutions:
D183*+G184*+R118K+N195F+R320K+R458K.

The variant is also disclosed in WO 01/66712. The alkaline alpha-amylase was produced in batch 03AGE014-4.

Alpha-amylase B is derived from a strain of Bacillus flavothermus and is disclosed in SEQ ID NO: 4.

Alpha-amylase C is derived from a strain of Bacillus licheniformis and is shown as SEQ ID NO: 6 in WO 99/19467.

Protease E is a Bacillus clausii (old name: Bacillus lentus C360=NCIB 10309) subtilisin having a M222S substitution covered by EP patent no. 396,608-B1 (Available on request from Novozymes, Denmark).

Lipase A is a lipase variant derived from Humicola lanuginosa strain DSM 4109 having the following mutations: T231R, N233R disclosed in U.S. Pat. No. 6,939,702-B (Available on request from Novozymes).

Cellulase A is a multi-component cellulase from Humicola insolens (Available on request from Novozymes, Denmark).

### Bacterial Strains.

Bacillus subtilis obtained from ATCC 10774.
E. coli ATCC #11229 and ATCC #25922

### Biofilm Medium.

Tryptic Soy Broth (TSB, purchased from VWR, P/N DF0370-07) medium was prepared according to the manufacturer's instructions, then diluted to 5% with water. 2 ml of trace elements were added per liter.

### Agar.

Tryptic Soy Agar (TSA, purchased from VWR, P/N DF0369-17) was used as per the manufacturer's directions.

### Trace Element Solution.

Per liter: 1.5 g CaCl₂, 1.0 g FeSO₄7.H₂O, 0.35 g MnSO₄.2H₂O, 0.5 g, NaMoO₄.

### Stainless Steel Coupons.

Stainless steel coupons No. 304 were obtained from Metal Samples Company (Munford, Ala.).

### BioLC Ion Chromatography System.

The IC system consisted of the following components:

### GP50 Gradient Pump (P/N 059493)

### ED50A Electrochemical Detector (P/N 059499)

AS50 Temperature Controlled Autosampler (P/N 056565) Electrochemical Cell for Integrated Amperometry, complete with Gold Electrode and

### Ag/AgCl Reference Electrode (P/N 060386)

### Chromelion Data Control Software CHM-1-IC (P/N 060930)

### CDC Biofilm Reactor.

Purchased from Biosurface Technologies, Inc. (P/N CBR 90-2) complete with polycarbonate coupons (24 for each reactor, P/N RD 128-PC).

### Detergent Cleaner Base.

Obtained from Weiman Products (IL, USA) as Burnishine ME—multiple enzyme detergent. The enzymes were denatured prior to use by heating in a microwave on high setting for 1 minute.

### Methods:

### Alpha-Amylase Activity (KNU)

The amylolytic activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e. at 37° C.+/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

### Determination of Degree of Identity Between Two Sequences

For purposes of the present invention, the degree of identity between two amino acid sequences is determined by the Clustal method (Higgins, 1989, CABIOS 5: 151-153) using the LASERGENE™ MEGALIGN™ software (DNASTAR, Inc., Madison, Wis.) with an identity table and the following multiple alignment parameters: Gap penalty of 10, and gap length penalty of 10. Pairwise alignment parameters were Ktuple=1, gap penalty=3, windows=5, and diago-nals=5.

### EXAMPLES

### Example 1

### Biofilm Removal Using Alpha-Amylase A and Alpha-Amylase C

Biofilm reactors consisted of a 400 ml beaker, a magnetic stirrer, and 2 stainless steel coupons. The coupons are taped vertically to the sides of the beaker so that the bottom edge of the coupon rested on the bottom of the beaker. A stir bar is added and the beakers are covered with a circle of aluminum foil and autoclaved. 200 ml of sterile biofilm medium is added to each beaker. To prepare the inoculum, each bacterial strain (from Bacillus subtilis) is grown overnight at 28° C. on plate count agar. Using a sterile swab, each is suspended in sterile water to an OD₆₈₆ of 0.100 and then diluted additionally to 10⁻¹. Each assay consisted of 4 control beakers without enzyme, 2 beakers with 50 mg of enzyme protein per liter of solution, and 2 beakers with 100 mg of enzyme protein per liter of solution. Beakers are first incubated at 37° C. overnight with stirring to grow the biofilm on the stainless steel coupons. Following this incubation step, the enzymes are added in the 2 dosages noted above as per the table below and incubated for an additional 2 hours at 40° C. Thereafter, each beaker and stainless steel coupon is rinsed carefully with sterile water, stained with crystal violet, rinsed with sterile water, the remaining biofilm solubilized with acetic acid, and the absorbance of an aliquot of each solution is measured at 600 nm using a spectrophotometer. The measured absorbances of the solutions provide a direct indication of the amount of biofilm remaining on the stainless steel coupons. A low absorbance corresponds to a good enzyme effect and little remaining biofilm, whereas a high absorbance corresponds to a poor (or lack thereof) enzyme effect and considerable remaining biofilm.

| Sample # | Enzyme Used | Enzyme Protein Conc. |
|---|---|---|
| 1 | Alpha-amylase A | 50 mg per L & 100 mg per L |
| 2 | Alpha-Amylase C | 50 mg per L & 100 mg per L |
| 3 | No enzyme (control) | NA |

### Example 2

### Raw Starch Solubilization Using Alpha-Amylase A, B, and C

The rate at which various alpha-amylases solubilize raw, unhydrated wheat starch was measured. Alpha-Amylase A, B and C, respectively, were used in the study.

Twenty five milliliters of a 1% raw wheat starch solution with pH 8 tris buffer and 15° dH was poured into a tube with lid and placed in a 40° C. water bath. The starting level of “reducing ends” was measured prior to addition of enzyme. The enzyme concentration used in the study was 3 mg enzyme protein per g raw wheat starch. One milliliter samples were taken out at different times. Twenty microliters of 1 M HCl was added prior to incubation at 99° C. for 10 minutes. The combination of acid and heat inactivates the amylase. Then 20 microL 1M NaOH was added to make sure the sample was no longer acidic. The sample was then diluted, incubated with color reagent (PHABH, potassium sodium tartrate, NaOH) at 95° C. for 10 minutes and finally centrifuged before measuring the OD at 410 nm on the supernatant. The control (100% hydrolyzed starch) was made by incubating a solution of 1% raw wheat starch in 1M HCl in an oven at 110° C. for 4 hours. This treatment was used to calculate the maximum amount of glucose which could be produced per gram of the raw wheat starch. This value was set to 100% in the graph shown in FIG. 1.

For Alpha-Amylase A and B it can be seen that the initial rate of raw wheat starch solubilization observed within the first 5 hours is significantly more rapid than for Alpha-Amylase C. This resulted in a greater percentage of the starch being solubilized by the former two alpha-amylases vs. for the latter over this time period.

### Example 3

### Biofilm Removal Using Alpha-Amylase A and C in Combination with Protease E and Detergent

A mono-component biofilm of Escherichia coli (ATCC #11229) is grown on polycarbonate coupons in a pre-sterilized CDC biofilm reactor. At the start of the experiment, cultures of E. coli were grown on tryptic soy agar (TSA) overnight at 37° C. The next morning, a single colony was picked from the plate using a 1 microL sterile inoculation loop and added to a solution of 40 g TSB/liter of water. This solution was incubated at 37° C. overnight to grow up the culture. The following day, 1 milliliter of this culture was added to 400 milliliters of minimal media (0.30 g TSB/liter sterile water) contained in the CDC biofilm reactor. The solution was slowly stirred at 130 rpm and grown for 2 days at 22° C. in a non fed batch mode. After the 2 day growth period, the coupon holder rods and coupons were removed from the reactor, rinsed in sterile dilution water to remove planktonic cells, the coupons were carefully removed from the rods and then incubated at 40° C. for 1 hour in the following solutions (30 milliliters each).
A: Detergent cleaner base alone, 0.21 g detergent in sterile water
B: Detergent cleaner base, 0.21 g+0.51 mg enzyme protein Protease E+0.06 mg enzyme protein Alpha-amylase A
C: Detergent cleaner base, 0.21 g+0.51 mg enzyme protein Protease E+0.16 mg enzyme protein Alpha-amylase C.

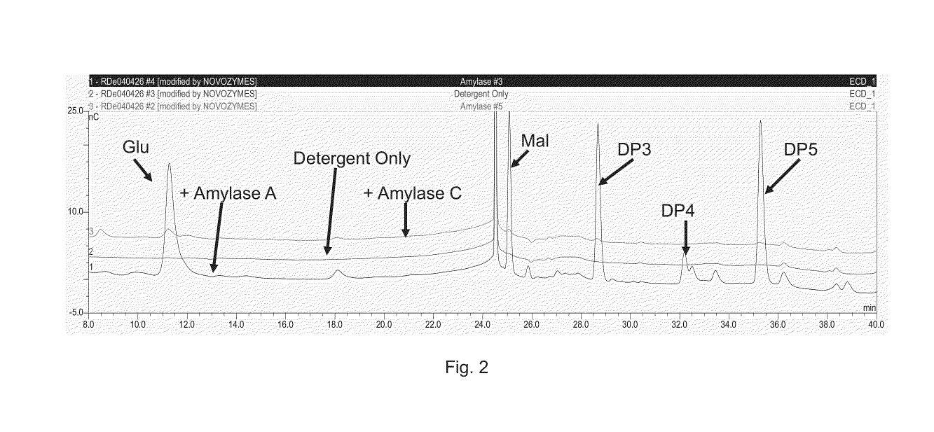

Following the incubation step, the coupons were removed. The solutions were filtered through 0.45□micro m Nylon syringe filters and their sugar contents measured by Ion Chromatography. PA100 guard and analytical columns (P/N 043055) were used for the separation. A mobile phase gradient between 60/40 deionized water/100 mM NaOH and 100% 100 mM NaOH/1M Sodium Acetate (exponential gradient started 10 minutes into the separation and ended at 85 minutes) was used to effect the separation. FIG. 2 shows an overlay of the 3 chromatograms generated in this experiment. When Alpha-Amylase A is used, a significantly higher level of low molecular weight sugars (glucose=glu, maltose=mal, maltotriose=DP3, maltotetraose=DP4, maltopentaose=DP5) were generated versus with the detergent alone or with Alpha-Amylase C. This indicated an increased level of biofilm removal through increased breakdown of the amylopectin exopolysaccharides produced by the E coli bacteria.

### Example 4

### Biofilm Removal Using Alpha-Amylase A and C in Combination with Protease E, Cellulase A, Lipase A and Detergent

Two CDC biofilm reactors fitted with polycarbonate coupons were autoclaved and filled with sterile 1/10 strength tryptic soy broth (TSB, 3g/liter strength) and inoculated with 1 ml log phase culture of Escherichia coli (ATCC #25922). The initial cell count in the reactors averaged to 5×10⁸ cfu/mL. Both reactors were operated for 24 hours in batch mode at 37° C. (no inflow or outflow). After this period, continuous flow of the 1/10 TSB was started at a flowrate of 12 ml/min at 37° C. The E. coli biofilm was grown for 4 days. After this time, 1 rod from each reactor (labeled as reactor 1 or 2) was pulled and placed into sterile glass beakers containing two hundred milliliters of the following filter-sterilized solutions.

A: Detergent cleaner base alone, 1.4 g detergent in sterile water
B: Detergent cleaner base, 1.4 g+3.4 mg enzyme protein Protease E+0.48 mg enzyme protein Lipase A+0.23 mg enzyme protein Cellulase A+0.40 mg enzyme protein Alpha-amylase A
C: Detergent cleaner base, 1.4 g+3.4 mg enzyme protein Protease E+0.48 mg enzyme protein Lipase A+0.23 mg enzyme protein Cellulase A+0.40 mg enzyme protein Alpha-amylase C.

The solutions in each of the beakers were incubated at 40° C. with moderate stirring for 30 minutes, after which each of the rods were gently rinsed in sterile water. Finally, E. coli was enumerated on 2 of the 3 coupons from each rod using tryptic soy agar (TSA). The average plate count results obtained from the study were as follows:

| | Average |
|---|---|
| | log₁₀ cfu/cm² |
| Treatment | on coupons |
| A | 4 × 10⁷ |
| B | 3 × 10⁴ |
| C | 2 × 10⁵ |

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present to disclosure including definitions will control.

Various references are cited herein, the disclosures of which are incorporated by reference in their entireties.

1. A method for preventing, removing, reducing or disrupting biofilm present on a surface, comprising contacting the surface with a biofilm control solution comprising an effective amount of alpha-amylase having at least 90% sequence identity to SEQ ID NO: 2, wherein the alpha-amylase enzyme is present in an amount of 0.01-100 mg of enzyme protein per L biofilm control solution, and wherein the biofilm is a microbially-produced biofilm including alpha-1,4 glucosidic linkages.

2. The method of claim 1, wherein the alpha-amylase is a Bacillus alpha-amylase.

3. The method of claim 2, wherein the alpha-amylase has at least 99% sequence identity to SEQ ID NO: 2.

4. The method of claim 2, wherein the alpha-amylase has a deletion in positions D183 and/or G184 (using SEQ ID NO: 2 for numbering).

5. The method of claim 4, wherein the alpha-amylase further has one or more of the following substitutions: R118K, N195F, R320K, R458K (using SEQ ID NO: 2 for numbering).

6. The method of claim 5, wherein the alpha-amylase has the following mutations: Delta (D183+G184)+R118K+N195F+R320K+R458K (using SEQ ID NO: 2 for numbering).

7. The method of claim 2, wherein the alpha-amylase has a substitution in position N195F (using SEQ ID NO: 2 for numbering).

8. The method of claim 1, wherein the alpha-amylase comprises Asn-Gly-Thr-Met-Met-Gln-Tyr-Phe-Glu-Trp in its N-terminal amino acid region.

9. The method of claim 1, wherein the surface is contacted for between 1 minute and 2 days.

10. The method of claim 1, which further comprises contacting the surface with a surfactant.

11. The method of claim 1, wherein the alpha-amylase is used in a concentration of between 0.005-500 mg enzyme protein.

12. The method of claim 1, wherein the alpha-amylase has a percentage (%) of hydrolyzed starch that is higher than 15 after 5 hours at 40° C., 3 mg enzyme protein per g starch, pH 8.0.

13. The method of claim 1, which further comprises contacting the surface with one or more additional enzymes selected from the group consisting of an aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidoreductases, pectinolytic enzyme, peptidoglutaminase, peroxidase, phytase, polyphenoloxidase, proteolytic enzyme, ribonuclease, transglutaminase, and xylanase.

14. The method of claim 1, which further comprises contacting the surface with one or more agents selected from the group consisting of dispersants, surfactants, anti-microbials, and biocides.

15. The method of claim 1, wherein the surface is a hard, soft, or porous surface.

16. The method of claim 15, wherein the surface is a membrane.

17. The method of claim 1, wherein the biofilm removal is done at a temperature between 10-70° C.

18. The method of claim 1, wherein the alpha-amylase consists of the mature alpha-amylase of SEQ ID NO:2.


SEQUENCE LISTING (6 sequences)

SEQ ID NO: 1 - DNA, 1455 bp, Bacillus sp.
  CDS (1)..(1455): AA560
caccataatggtacgaacggcacaatgatgcagtactttgaatggtatctaccaaatgacggaaaccattggaatagattaaggtctgatgcaagtaacctaaaagataaagggatctcagcggtttggattcctcctgcatggaagggtgcctctcaaaatgatgtggggtatggtgcttatgatctgtatgatttaggagaattcaatcaaaaaggaaccattcgtacaaaatatggaacgcgcaatcagttacaagctgcagttaacgccttgaaaagtaatggaattcaagtgtatggcgatgttgtaatgaatcataaagggggagcagacgctaccgaaatggttagggcagttgaagtaaacccgaataatagaaatcaagaagtgtccggtgaatatacaattgaggcttggacaaagtttgactttccaggacgaggtaatactcattcaaacttcaaatggagatggtatcactttgatggagtagattgggatcagtcacgtaagctgaacaatcgaatttataaatttagaggtgatggaaaagggtgggattgggaagtcgatacagaaaacggtaactatgattacctaatgtatgcagatattgacatggatcacccagaggtagtgaatgagctaagaaattggggtgtttggtatacgaatacattaggccttgatggttttagaatagatgcagtaaaacatataaaatacagctttactcgtgattggattaatcatgttagaagtgcaactggcaaaaatatgtttgcggttgcggaattttggaaaaatgatttaggtgctattgaaaactatttaaacaaaacaaactggaaccattcagtctttgatgttccgctgcactataacctctataatgcttcaaaaagcggagggaattatgatatgaggcaaatatttaatggtacagtcgtgcaaagacatccaatgcatgctgttacatttgttgataatcatgattcgcaacctgaagaagctttagagtcttttgttgaagaatggttcaaaccattagcgtatgctttgacattaacacgtgaacaaggctacccttctgtattttatggagattattatggcattccaacgcatggtgtaccagcgatgaaatcgaaaattgacccgattctagaagcgcgtcaaaagtatgcatatggaagacaaaatgactacttagaccatcataatatcatcggttggacacgtgaagggaatacagcacaccccaactccggtttagctactatcatgtccgatggggcaggaggaaataagtggatgtttgttgggcgtaataaagctggtcaagtttggaccgatatcactggaaatcgtgcaggtactgttacgattaatgctgatggatggggtaatttttctgtaaatggaggatcagtttctatttgggtaaacaaa

SEQ ID NO: 2 - protein, 485 aa, Bacillus sp.
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 3 - DNA, 1860 bp, Bacillus flavothermus
  CDS (1)..(1857)
  sig_peptide (1)..(99)
  mat_peptide (100)..(1857)
  misc_feature (100)..(1551): catalytic domain
atgtccctattcaaaaaaagctttccgtggattttatccctacttcttttgttttcgtttattgctcctttttccattcaaacagaaaaagtccgagctggaagtgtgccggtaaatggcacaatgatgcaatatttcgaatggtaccttccagacgatggaacactatggacgaaagtagcaaataacgctcaatctttagcgaatcttggcattactgccctttggcttccccctgcctataaaggaacaagcagcagtgacgttggatatggcgtttatgatttatatgaccttggagagtttaatcaaaaaggaactgtccgaacaaaatacgggacaaaaacacaatatatccaagcaatccaagcggcgcatacagcagggatgcaagtatatgcagatgtcgtctttaaccataaagccggtgcagatggaacagaactagtcgatgcagtagaagtaaatccttctgaccgcaatcaagaaatatcaggaacatatcaaatccaagcgtggacaaaatttgattttcctggtcgtggaaacacctattctagttttaaatggcgttggtatcatttcgatggaacggactgggatgagagtagaaaactaaatcgtatttacaagttccgcggcacgggaaaagcatgggattgggaagtagatacagaaaacgggaattatgactatctcatgtatgcagatttagatatggatcatccagaggttgtatccgaactaaaaaattggggaaagtggtatgtaaccacaaccaatatcgacggattccgtctggatgcagtgaagcatattaaatatagctttttcccggactggctatcgtacgtacgaacccaaacacaaaagcctctttttgccgttggggaattttggagctatgacattagcaagttgcacaactatattacaaagacgaacggctctatgtccctattcgatgccccgctgcataacaatttttatatagcatcgaaatcaggcggttattttgatatgcgcacattactcaacaacacattgatgaaagatcagcctacattagcagtcacattagtggataatcacgatactgagccagggcaatctctgcagtcatgggtcgagccatggtttaaaccgttagcttacgcatttatcttgacccgccaagaaggttatccttgcgtcttttatggagattactatggtattccaaaatacaacattcctgcgctgaaaagcaaacttgatccgctgttaattgccagaagagattatgcctatggaacacagcacgactatattgacagtgcggatattatcggttggacgcgggaaggagtggctgaaaaagcaaattcaggactggctgcactcattaccgacgggcctggcggaagcaaatggatgtatgttggaaaacaacacgctggcaaaacgttttatgatttaaccggcaatcgaagtgatacagtgacaatcaatgctgatggatggggagaatttaaagtcaatggagggtctgtatccatatgggttccaaaaatatcaaccacttcccaaataacatttactgtaaataacgccacaaccgtttggggacaaaatgtatacgttgtcgggaatatttcgcagctggggaactgggatccagtccacgcagttcaaatgacgccgtcttcttatccaacatggactgtaacaatccctcttcttcaagggcaaaacatacaatttaaatttatcaaaaaagattcagctggaaatgtcatttgggaagatatatcgaatcgaacatacaccgtcccaactgctgcatccggagcatatacagccagctggaacgtgccctag

SEQ ID NO: 4 - protein, 619 aa, Bacillus flavothermus
MSLFKKSFPWILSLLLLFSFIAPFSIQTEKVRAGSVPVNGTMMQYFEWYLPDDGTLWTKVANNAQSLANLGITALWLPPAYKGTSSSDVGYGVYDLYDLGEFNQKGTVRTKYGTKTQYIQAIQAAHTAGMQVYADVVFNHKAGADGTELVDAVEVNPSDRNQEISGTYQIQAWTKFDFPGRGNTYSSFKWRWYHFDGTDWDESRKLNRIYKFRGTGKAWDWEVDTENGNYDYLMYADLDMDHPEVVSELKNWGKWYVTTTNIDGFRLDAVKHIKYSFFPDWLSYVRTQTQKPLFAVGEFWSYDISKLHNYITKTNGSMSLFDAPLHNNFYIASKSGGYFDMRTLLNNTLMKDQPTLAVTLVDNHDTEPGQSLQSWVEPWFKPLAYAFILTRQEGYPCVFYGDYYGIPKYNIPALKSKLDPLLIARRDYAYGTQHDYIDSADIIGWTREGVAEKANSGLAALITDGPGGSKWMYVGKQHAGKTFYDLTGNRSDTVTINADGWGEFKVNGGSVSIWVPKISTTSQITFTVNNATTVWGQNVYVVGNISQLGNWDPVHAVQMTPSSYPTWTVTIPLLQGQNIQFKFIKKDSAGNVIWEDISNRTYTVPTAASGAYTASWNVP

SEQ ID NO: 5 - DNA, 1920 bp, Bacillus licheniformis
  CDS (421)..(1872)
cggaagattggaagtacaaaaataagcaaaagattgtcaatcatgtcatgagccatgcgggagacggaaaaatcgtcttaatgcacgatatttatgcaacgttcgcagatgctgctgaagagattattaaaaagctgaaagcaaaaggctatcaattggtaactgtatctcagcttgaagaagtgaagaagcagagaggctattgaataaatgagtagaagcgccatatcggcgcttttcttttggaagaaaatatagggaaaatggtacttgttaaaaattcggaatatttatacaacatcatatgtttcacattgaaaggggaggagaatcatgaaacaacaaaaacggctttacgcccgattgctgacgctgttatttgcgctcatcttcttgctgcctcattctgcagcagcggcggcaaatcttaatgggacgctgatgcagtattttgaatggtacatgcccaatgacggccaacattggaggcgtttgcaaaacgactcggcatatttggctgaacacggtattactgccgtctggattcccccggcatataagggaacgagccaagcggatgtgggctacggtgcttacgacctttatgatttaggggagtttcatcaaaaagggacggttcggacaaagtacggcacaaaaggagagctgcaatctgcgatcaaaagtcttcattcccgcgacattaacgtttacggggatgtggtcatcaaccacaaaggcggcgctgatgcgaccgaagatgtaaccgcggttgaagtcgatcccgctgaccgcaaccgcgtaatttcaggagaacacctaattaaagcctggacacattttcattttccggggcgcggcagcacatacagcgattttaaatggcattggtaccattttgacggaaccgattgggacgagtcccgaaagctgaaccgcatctataagtttcaaggaaaggcttgggattgggaagtttccaatgaaaacggcaactatgattatttgatgtatgccgacatcgattatgaccatcctgatgtcgcagcagaaattaagagatggggcacttggtatgccaatgaactgcaattggacggtttccgtcttgatgctgtcaaacacattaaattttcttttttgcgggattgggttaatcatgtcagggaaaaaacggggaaggaaatgtttacggtagctgaatattggcagaatgacttgggcgcgctggaaaactatttgaacaaaacaaattttaatcattcagtgtttgacgtgccgcttcattatcagttccatgctgcatcgacacagggaggcggctatgatatgaggaaattgctgaacggtacggtcgtttccaagcatccgttgaaatcggttacatttgtcgataaccatgatacacagccggggcaatcgcttgagtcgactgtccaaacatggtttaagccgcttgcttacgcttttattctcacaagggaatctggataccctcaggttttctacggggatatgtacgggacgaaaggagactcccagcgcgaaattcctgccttgaaacacaaaattgaaccgatcttaaaagcgagaaaacagtatgcgtacggagcacagcatgattatttcgaccaccatgacattgtcggctggacaagggaaggcgacagctcggttgcaaattcaggtttggcggcattaataacagacggacccggtggggcaaagcgaatgtatgtcggccggcaaaacgccggtgagacatggcatgacattaccggaaaccgttcggagccggttgtcatcaattcggaaggctggggagagtttcacgtaaacggcgggtcggtttcaatttatgttcaaagatagaagagcagagaggacggatttcctgaaggaaatccgtttttttatttt

SEQ ID NO: 6 - protein, 483 aa, Bacillus licheniformis [sequence omitted]